SEQUENCE LISTING (14 sequences)

SEQ ID NO: 1 - DNA, 1331 bp, Arabidopsis thaliana
ccatcacggatccgcatgctcccggccgccatggcggccgcgggaattcgattggggcatgcatgtctgaaaacagagaggcttggtcagcgaattccgaggaaatggagctgcttcacggctccaacagattatcttcaccggaacatgttcgtagaagagtctccgggaattcttctgaggacggttcgcctcgaatttgccggcagcagtcgttcggtcgcgacataggccatgcagctgccgagacgtatctcattactcgacttagcttcaaccttcttggatatctcggggtaggttaccggtggatcacaagattacttgctcttgcttgttatgcaatgctccttatgcctggtttccttcaagttgcatatctttattttttctcatctcaagtcaggaggagtatagtttatggaggtcatccaaggaataggctggatttgtacataccgccaacgagtgatggcctgaagccggttgtagttttcgttactggtggagcttggattatcggttacaaagcttggggttctctcttgggactacagctagcagaaagggacataattgtggcatgtctagattacagaaactttcctcaggggacaattagtgatatggtaagtgatgcagctcaaggaatctcatttgtctgcaataacatctctgcatttggaggtgatcctaacagaatctatctgatggggcaatcggctggtgcacatatctcttcttgtgctctctttgaacaagctattaaagaatcaagaggggagagcatctcatggagtgtatctcagattaaagcttactttggtttatctggagggtacaatctctttaatctggttgaacacttccacaaccgaggcttgtatcgttcaattttcctaagcataatggaaggagaagagtcctttaaacaattctcccctgaagtaagattgaaagacctgaatgtaagaaaagctgcagccctcttacctcatattatactctttcatggatccgcagattattcaatcccacctgaagcaagcaaaacttttacagatgctcttcaagctgctgaagtgaaagctgagctagttatgtacaagggtaaaactcataccgacctatttcttcaggatcctctgagaggcggtaaagatgagctatttgatcatatagtctctatgatacacgccgatgacagtgacgcattgagaaacgatgctgttgcgccaccgaggaaacggcttgttccagagttcttgctgaaactggctggcagggtcagccctttttgagtcgacctgcagccaagcttaattagctga

SEQ ID NO: 2 - protein, 412 aa, Arabidopsis thaliana
MSENREAWSANSEEMELLHGSNRLSSPEHVRRRVSGNSSEDGSPRICRQQSFGRDIGHAAAETYLITRLSFNLLGYLGVGYRWITRLLALACYAMLLMPGFLQVAYLYFFSSQVRRSIVYGGHPRNRLDLYIPPTSDGLKPVVVFVTGGAWIIGYKAWGSLLGLQLAERDIIVACLDYRNFPQGTISDMVSDAAQGISFVCNNISAFGGDPNRIYLMGQSAGAHISSCALFEQAIKESRGESISWSVSQIKAYFGLSGGYNLFNLVEHFHNRGLYRSIFLSIMEGEESFKQFSPEVRLKDLNVRKAAALLPHIILFHGSADYSIPPEASKTFTDALQAAEVKAELVMYKGKTHTDLFLQDPLRGGKDELFDHIVSMIHADDSDALRNDAVAPPRKRLVPEFLLKLAGRVSPF

SEQ ID NO: 3 - DNA, 1155 bp, Arabidopsis thaliana
atggcatcttcactgaagaagcttatctcaagctttctacttgtcttatactccaccaccatcattgttgcttcatcagaatctcgatgtaggcgttttaaatcgatcatcagctttggtgattccatcgcagacaccggaaattatctccatctctctgatgtcaatcaccttcctcaatccgcctttcttccttatggcgaaagcttcttccatcctccctccggtcgtgcctctaatggccgtctcatcatcgacttcattgccgaattcttgggactaccatacgtaccgccttactttggatcccaaaacgttagcttcgaacaagggatcaattttgcggtgtatggagcaaccgcattggaccgagcgtttcttttgggaaaaggaattgaatctgatttcaccaatgttagtttaagtgttcagcttgacaccttcaagcagattttgcctaacttatgcgcctcgtctactcgtgattgcaaagagatgcttggagactcgctgatactcatgggagagattggaggaaacgactataattacccattttttgaaggaaaaagtatcaatgaaatcaaagagcttgttcctctaatcgtcaaagctatttcttctgctattgtggatttgattgatttagggggcaaaacatttttggtacccggaggcttcccaacaggatgttctgcggcgtatcttactctatttcagaccgtggcagaaaaagaccaggaccctttaacaggttgttacccattgctcaacgaatttggcgagcaccacaacgaacagctaaagacagaactcaagcgactccaaaaattctatcctcatgtcaacatcatttacgctgactaccacaactccttataccggttttatcaagaaccagctaaatacgggtttaagaacaaacctttagctgcttgctgtggagtcggaggtaaatacaacttcactattggtaaggagtgtggatacgaaggagttaactattgtcaaaatccttctgagtatgtgaactgggatggttatcatttaaccgaagccgcctaccagaagatgactgagggtatactcaacggtccctatgcaactcctgctttcgactggtcctgccttggctctggtacagtggatacataa

SEQ ID NO: 4 - protein, 384 aa, Arabidopsis thaliana
MASSLKKLISSFLLVLYSTTIIVASSESRCRRFKSIISFGDSIADTGNYLHLSDVNHLPQSAFLPYGESFFHPPSGRASNGRLIIDFIAEFLGLPYVPPYFGSQNVSFEQGINFAVYGATALDRAFLLGKGIESDFTNVSLSVQLDTFKQILPNLCASSTRDCKEMLGDSLILMGEIGGNDYNYPFFEGKSINEIKELVPLIVKAISSAIVDLIDLGGKTFLVPGGFPTGCSAAYLTLFQTVAEKDQDPLTGCYPLLNEFGEHHNEQLKTELKRLQKFYPHVNIIYADYHNSLYRFYQEPAKYGFKNKPLAACCGVGGKYNFTIGKECGYEGVNYCQNPSEYVNWDGYHLTEAAYQKMTEGILNGPYATPAFDWSCLGSGTVDT

SEQ ID NO: 5 - DNA, 1293 bp, Arabidopsis thaliana
atgtgtagaagatatttcgtgcatagttgttttttacttctgctgttactcaactatttgccctttcgatttaacttctcttataatcttgataggtatccatatgaagctattcgtgttgttacatctgatggatatggtcttctcttggaaaggataccaagacgagatgcgaggaaagctgtttatttgcagcatggtgtaatggattcttcaatgggatgggtatcaaatggtgttgttggatcaccagcttttgcagcttatgatcaaggctacgatgttttcctagggaactttcgtggtttagtttcaagagatcatgtgaaaaaaaacatatcctcaaaagatttctggcgttactccatcaatgaacatgcaacggaagatatcccagcaatgatagagaagattcacgaaatcaaaacttcagaactgaaactttatcagcctactatggaagaggtagtaaacgaggatcaaccgtataagctttgtgttgtatctcacagtttaggcggtgccgcggttctgatgtatgtaatcacccgtaaaatcgaagagaaaccgcacagactctcgagactgatccttctttcgcctgctgggtttcactatgactccaacatgtgtttcacgttgatggagtacacgttccttttcttgggtcctgtactatctcggattgttcctgctttctacatccccactaaattcttccggatgcttctcaacaagttagctcgagatttccataactatcctgctgttggtggattagtccaaacattgatgagttatgtagttggcggagatagctcaaactgggttggagtcatgggattgcctcactataacatgaacgatatgccaggtatatccttccgtgtggctcagcatcttgcacagattaaacatagcggtaagttcaagatgtttgattacggtagcagttcagctaatatggatgtttacgggtcccctgagccgctcgatcttggggagttttatgggttgatcgatgtgcctgtggatttagtagctggaaagaaagacaaagtgattagaccttcaatggttaggaaacattacagggtgatgagagattcaggagtagatgtttcttacaatgagtttgagtatgctcatttggattttaccttctctcaccgtgaagagcttttggcgtatgtgatgtcgcggttacttctcgtggagccaacgcagactcagacggttcataagaaggggatgaagctgaagaagaaaatggaaacaggcaaacctcatctg

SEQ ID NO: 6 - protein, 431 aa, Arabidopsis thaliana
MCRRYFVHSCFLLLLLLNYLPFRFNFSYNLDRYPYEAIRVVTSDGYGLLLERIPRRDARKAVYLQHGVMDSSMGWVSNGVVGSPAFAAYDQGYDVFLGNFRGLVSRDHVKKNISSKDFWRYSINEHATEDIPAMIEKIHEIKTSELKLYQPTMEEVVNEDQPYKLCVVSHSLGGAAVLMYVITRKIEEKPHRLSRLILLSPAGFHYDSNMCFTLMEYTFLFLGPVLSRIVPAFYIPTKFFRMLLNKLARDFHNYPAVGGLVQTLMSYVVGGDSSNWVGVMGLPHYNMNDMPGISFRVAQHLAQIKHSGKFKMFDYGSSSANMDVYGSPEPLDLGEFYGLIDVPVDLVAGKKDKVIRPSMVRKHYRVMRDSGVDVSYNEFEYAHLDFTFSHREELLAYVMSRLLLVEPTQTQTVHKKGMKLKKKMETGKPHL

SEQ ID NO: 7 - DNA, 33 bp, artificial sequence
  description of the artificial sequence oligonucleotide primer
cccgcatgcatgcagttgtctccggaacgatgc

SEQ ID NO: 8 - DNA, 33 bp, artificial sequence
  description of the artificial sequence oligonucleotide primer
ggggcatgcatgtctgaaaacagagaggcttgg

SEQ ID NO: 9 - DNA, 33 bp, artificial sequence
  description of the artificial sequence oligonucleotide primer
aaagcatgcatggagctgcttcacggctccaac

SEQ ID NO: 10 - DNA, 34 bp, artificial sequence
  description of the artificial sequence oligonucleotide primer
aaagtcgactcaaaaagggctgaccctgccagcc

SEQ ID NO: 11 - DNA, 30 bp, artificial sequence
  description of the artificial sequence oligonucleotide primer
ccccgcatgcatggcttcttcactgaagaa

SEQ ID NO: 12 - DNA, 36 bp, artificial sequence
  description of the artificial sequence oligonucleotide primer
aaactgcagttatgtatccactgtaccagagccaag

SEQ ID NO: 13 - DNA, 36 bp, artificial sequence
  description of the artificial sequence oligonucleotide primer
ggggcatgcatgtgtagaagatatttcgtgcatagt

SEQ ID NO: 14 - DNA, 34 bp, artificial sequence
  description of the artificial sequence oligonucleotide primer
tttctgcagtgacagatgaggtttgcctgtttcc

(19) United States
(12) Patent Application Publication
(10) Pub. No.: US 2003/0154515 A1
(43) Pub. Date: Aug. 14, 2003

(54) Triacylglycerol lipases

(76) Inventors: Martina Korner, Berlin (DE); Ekkehart Berndt, Obertshausen (DE); Kathrin Fritsche, Arnhem (NL); Ivo Feussner, Gottingen (DE)

Correspondence Address: KENYON & KENYON, 1500 K STREET, N.W., SUITE 700, WASHINGTON, DC, 20005, US

(21) Appl. No.: 10/304,038

(22) Filed: Nov. 26, 2002

Related U.S. Application Data
(63) Continuation-in-part of application No. PCT/EP01/06156, filed on May 30, 2001.

(30) Foreign Application Priority Data
May 31, 2000 (DE) 10026845.5

(51) Int. Cl. A01H 1/00; C12N 15/82; C12N 9/04

(52) U.S. Cl. 800/281; 435/468; 435/190

(57) ABSTRACT

The invention relates to nucleic acid molecules, which code for proteins with the enzymatic activity of an acyl hydrolase, in particular, a triacylglyercol lipase (TAG-lipase) and method for the production of transgenic plants, which contain said nucleic acid molecules and whose acyl hydrolase content, in particular of TAG-lipase is altered in comparison to wild type plants. The invention further relates to said novel plants, the parts, products and plant cells thereof and the use of the nucleic acid molecules to influence the content of poylunsaturated fatty acids in transgenic plants.

This is a continuation-in-part of International Application PCT/EP01/06156, with an international filing date of May 30, 2001, published in German, which claims priority to German Application 10026845.5, filed on May 31, 2000.

### FIELD OF THE INVENTION

The invention relates to nucleic acid molecules which code for proteins having the enzymatic activity of an acyl hydrolase, in particular of a triacylglycerol lipase (TAG lipase) and methods for the production of transgenic plants containing said nucleic acid molecules and whose acyl hydrolase content, in particular whose TAG lipase content, is altered in comparison with wild-type plants. The invention further relates to said novel plants, parts, products and plant cells thereof and the use of the nucleic acid molecules for influencing the content of poly-unsaturated fatty acids in transgenic plants.

### BACKGROUND OF THE INVENTION

Lipases catalyse a large number of reactions, many of which have industrial potential. TAG lipases catalyse the conversion of triacylglycerol and water into diacylglycerol and a fatty acid anion.

The amino acid sequences of lipases from the human stomach, from the rat tongue and from the human hepatic lysosome are homologous, but except for a sequence of six amino acids, including the important amino acid serine 152 of the lipase from swine pancreas (M. W. Bodner, (1987) Biochem. Biophys. Acta 909:237-244), they are not related to the lipase from the swine pancreas. These enzymes are glycosylated, contain a hydrophobic leader peptide and belong to the family of acid lipases (D. Ameis et al. (1994) Euro. J. Biochem. 219:905-914). The lysosomal acid lipase (LAL) is an essential hydrolase in the intracellular degradation of cholesteryl esters and triacylglycerols and plays a role in the mobilization of the seed oil during germination.

Neutral triacylglycerol lipases have extensively been studied in fungi, bacteria, mammals and insects. Nucleotide sequences have been described which show similarities to the neutral triacylglycerol lipases in Arabidopsis thaliana and Ipomea nil, but their function has not been demonstrated. Furthermore, the crystal structure of the triacylglycerol lipase from Mucor miehei has been reported, where a trypsin-like catalytic triad Ser-His-Asp with an active serine residue underneath a short helical fragment of a long surface loop was found (L. Brady et al. (1990) Nature 343:767-770).

It is assumed that the main physiological function of the plant TAG lipases is to mobilize storage lipids during the germination process. Especially for oil seed plants, the storage lipids are the main carbon source for the growing seedling. Certain plant lipoxygenases (LOXs) can be detected at the membrane of the lipid body of different oil seed plants during germination. They specifically convert polyene fatty acid residues, i.e. poly-unsaturated fatty acid residues, from triacylglycerols directly into hydroperoxy or ketodiene fatty acid derivatives. Metabolites resulting from this reaction, i.e. triacylglycerols, whose acyl residues consist of 13-hydro(pero)xylinoleic acid, are then hydrolysed by a TAG lipase, which is highly specific for oxidized polyene fatty acid residues. The fatty acids modified in such a way serve as an energy reserve for the growing seedling.

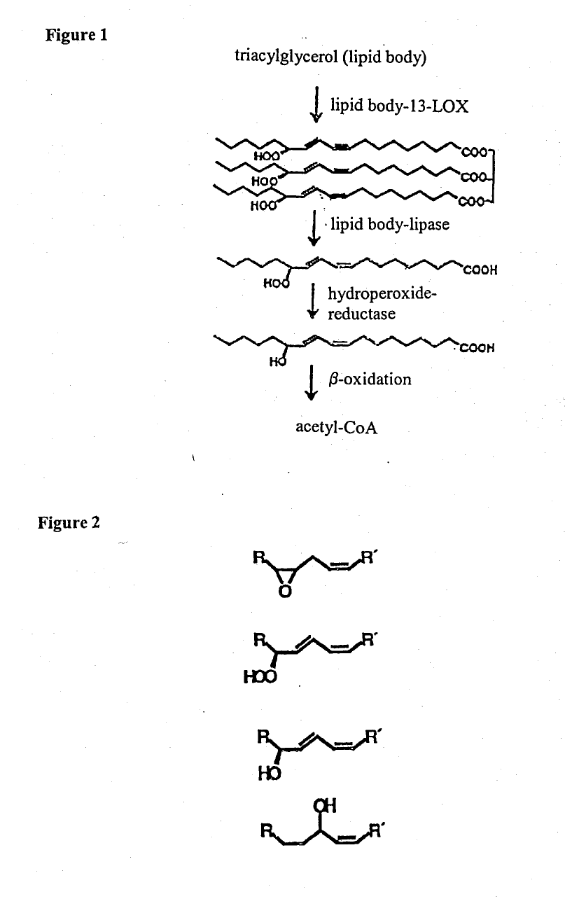

As already mentioned above, the occurrence of LOXs is accompanied by the accumulation of hydro(pero)xy derivatives, which, in contrast to the non-oxygenated poly-unsaturated fatty acid residues (PUFA residues) are cleaved from the storage lipids. From these observations it is concluded that the oxidation reaction catalysed by the LOXs initiates the catabolism of poly-unsaturated fatty acids (reductase pathway) in plants (see FIG. 1). Moreover, the specific TAG lipase was purified from germinating cucumber seedlings and its biochemical properties were determined. This lipid body-associated TAG lipase did in fact show a high specificity for oxidated poly-unsaturated fatty acids with the structural motifs shown in FIG. 2. This specific TAG lipase was also found in the lipid body fraction of developing seedlings, suggesting an at least double physiological function of these enzymes in plants.

The isolation of plant TAG lipases may be useful for processing plant oils containing fatty acids with unusual oxygenated structures. Such plant oils are problematic when used in the food industry, because the oxygenated fatty acids contained therein result in a shorter storage stability. Said fatty acid residues may be selectively removed from the corresponding oils by specific TAG lipases. The released oxygenated fatty acids are in turn important copolymers for the manufacture of plastics.

After production of the poly-unsaturated fatty acids, which are esterified in triacylglycerols, the in a defined manner structured lipids with oxygenated fatty acid residues in certain positions of the glycerol backbone may be purified by removal of the contaminating lipid peroxides. On the other hand, certain oxygenated fatty acids may be introduced into certain TAGs' positions. Transgenic plants, which accumulate large amounts of these oxygenated fatty acids within their seed oil, require this specific lipase for germination. In addition, the content of PUFAs in the seed oil may be reduced by the co-expression of a TAG-LOX and a TAG lipase in the seed.

### SUMMARY OF THE INVENTION

Thus, it is an essential object of the present invention to provide a method, by which the content of poly-unsaturated fatty acids, particularly of oxygenated poly-unsaturated fatty acids in plants, may be decreased or increased. A further object of the present invention is to provide nucleic acid molecules, which may be transferred to plant cells or plant seeds, to influence the content of polyene fatty acids, in particular of oxidized polyene fatty acid residues. Further objects of the present invention will become clear from the following description.

These problems are solved by the subject-matter of the independent claims, especially based on the provision of the DNA sequences according to the invention, whose gene products are directly involved in the hydrolysis of triacylglycerols with oxygenated, unsaturated fatty acid residues, and the transfer of the DNA sequences into plants, which results in a modified content of such fatty acids.

Advantageous embodiments are defined in the respective sub-claims.

The present invention thus comprises recombinant nucleic acid molecules, comprising

a) regulatory sequences of a promoter that is active in plant cells;

b) operatively linked thereto a DNA sequence, which codes for a protein having the enzymatic activity of an acyl hydrolase, in particular of a triacylglycerol lipase (TAG lipase), more preferably a TAG lipase which is specific for oxygenated polyene fatty acids; and

c) operatively linked thereto regulatory sequences, which may act as transcription, termination and/or polyadenylation signals in plant cells.

### DETAILED DESCRIPTION

Sequences that code for a TAG lipase have already been described in the state of the art, however without providing any evidence for their function. PCT/US99/09280 discloses inter alia the sequences of TAG lipase cDNA clones from maize, rice and soybean.

Surprisingly, the present invention for the first time succeeded in providing nucleic acid molecules, which code for a protein having the enzymatic activity of a TAG lipase. Furthermore, the present invention also describes for the first time the over-expression of a protein encoded by the above-mentioned nucleic acid molecules and having the activity of a TAG lipase in plants. The TAG lipases according to the invention show a high specificity for oxygenated polyene fatty acids, but also hydrolyse normal TAGs under certain reaction conditions.

In particular the invention pertains to plant DNA sequences that code for a protein having the biological activity of an acyl hydrolase, in particular a TAG lipase, or a biologically active fragment thereof from Arabidopsis. The invention more preferably relates to the sequences identified in SEQ ID NO: 1, SEQ ID NO: 3 and SEQ ID NO: 5 of the attached sequence listing, the derived amino acid sequences being identified in SEQ ID NO: 2, SEQ ID NO: 4 and SEQ ID NO: 6.

In the context of this invention biologically active fragment means that the mediated biological activity suffices to influence the content of poly-unsaturated fatty acids, particularly of poly-unsaturated oxygenated fatty acids.

The DNA sequence, which codes for a protein having the biological activity of an acyl hydrolase, particularly of a TAG lipase, more preferably of a TAG lipase which is specific for oxygenated polyene fatty acids, may be isolated from natural sources or synthesised by conventional methods. Using routine molecular biological techniques (see for example Sambrook et al. (2001) vide supra) it is possible to prepare and to produce the desired constructs for the transformation of plant cells. Methods for cloning, mutagenesis, sequence analysis, restriction analysis and other biochemical/molecular biological methods are well known to the person skilled in the art and are conventionally used for gene technological manipulation in prokaryotic cells. Thus, not only may suitable chimeric gene constructs containing the desired fusion of promoter sequences and TAG lipase DNA sequence of the present invention and optionally further regulatory and/or signal sequences be produced, but rather the person skilled in the art may further, if desired, introduce various mutations into the TAG lipase encoding DNA sequence according to the invention using routine techniques, thus resulting in the synthesis of proteins with possibly altered biological properties. By this, construction of deletion mutants is possible, in which by progressive deletion at the 5′-end or the 3′-end of the coding DNA sequence the synthesis of accordingly shortened proteins may be achieved. It is also possible to purposely produce enzymes, which are localised within specific compartments of the plant cell due to the addition of respective leader peptides. Such sequences are described in the literature and are well known to the person skilled in the art (see for example Braun et al. (1992) EMBO J. 11:3219-3227; Wolter F. et al. (1988) Proc. Natl. Acad. Sci. USA 85:846-850; Sonnewald U. et al. (1991) Plant J. 1:95-106). It is also conceivable to introduce point mutations at sites, where an alteration of the amino acid sequence influences, for example, the enzymatic activity or the regulation of the enzyme. By this strategy, for example mutants may be created, which are no longer susceptible to the regulatory mechanisms by means of allosteric or covalent modification, which normally prevail in the cell. Furthermore, mutants having an altered substrate or product specificity may be produced. Mutants having an altered activity, temperature and/or pH profile may also be produced.

For the gene technological manipulation in prokaryotic cells the recombinant nucleic acid molecules of the invention or parts thereof may be incorporated into plasmids, which allow mutagenesis or sequence alteration by recombining of DNA sequences. Using standard techniques (see for example Sambrook et al. (2001), vide supra) base exchanges may be created or natural or synthetic sequences may be added. For the fusion of the DNA fragments with each other adapters or linkers may be attached to the fragments, where necessary. Additionally, appropriate restriction sites may be provided or abundant DNA or restriction sites may be removed using enzymatic and other manipulation techniques. Where insertions, deletions or substitutions are to be carried out, in vitro mutagenesis, “primer repair,” restriction or ligation may be utilised. In general, for analysis purposes sequence analysis, restriction analysis and other biochemical/molecular biological methods are performed.

In a preferred embodiment the DNA sequence, which encodes a protein having the biological activity of an acyl hydrolase, particularly of a TAG lipase, more preferably of a TAG lipase specific for oxygenated polyene fatty acids is selected from the group, consisting of

a) DNA sequences comprising the nucleotide sequence identified in SEQ ID NO: 1,

b) DNA sequences comprising the nucleotide sequence identified in SEQ ID NO: 3,

c) DNA sequences comprising the nucleotide sequence identified in SEQ ID NO: 5,

d) DNA sequences comprising a nucleotide sequence that codes for the amino acid sequence identified in SEQ ID NO: 2 or fragments thereof,

e) DNA sequences that encode the amino acid sequence identified in SEQ ID NO: 4 or fragments thereof,

f) DNA sequences that encode the amino acid sequence identified in SEQ ID NO: 6 or fragments thereof,

g) DNA sequences comprising a nucleotide sequence, which hybridises to a complementary strand of the nucleotide sequence of a), b), c), d), e) or f), or fragments of said nucleotide sequence,

h) DNA sequences comprising a nucleotide sequence, which is degenerate to a nucleotide sequence of g), or fragments of said nucleotide sequence,

i) DNA sequences, which represent a derivative, analogue or fragment of a nucleotide sequence of a), b), c), d), e), f), g) or h).

In the context of the present invention the term “hybridisation” means hybridisation under conventional hybridisation conditions, preferably under stringent conditions, as described for example, in Sambrook et al. (2001), vide supra).

DNA sequences that hybridise to DNA sequences which code for a plant protein having the biological activity of a TAG lipase, particularly of a TAG lipase which is specific for oxygenated polyene fatty acids can, for example, be isolated from genomic or cDNA libraries of any plant, which is naturally in possession of the TAG lipase DNA sequences according to the invention. Such DNA sequences can be identified and isolated, for example, by using DNA sequences, which have exactly or substantially the nucleotide sequence identified in SEQ ID NO: 1, SEQ ID NO: 3 or SEQ ID NO: 5, or parts thereof, or the reverse complements of these DNA sequences, e.g. by hybridisation according to standard methods (see e.g. Sambrook et al. (2001), vide supra). Fragments used as a hybridisation probe may also be synthetic fragments produced by conventional DNA synthesis techniques and whose sequence is substantially identical to one of the afore-mentioned TAG lipase DNA sequences or a part thereof.

The term “hybridisation” refers to the formation of double stranded nucleic acid molecules which are also designated as “duplexes” from complementary single stranded nucleic acid molecules. Thus hybridisation may result in the formation of DNA-DNA-duplexes, DNA-RNA-duplexes or RNA-RNA-duplexes. Hybridisation experiments are usually used to determine sequence complementarity and thus identity between two nucleic acid molecules.

Hybridisation of nucleic acid-sequences may take place in vivo under cellular conditions or in vitro. According to the invention, a hybridisation in vivo is to be carried out under conditions that are stringent enough in order to ensure a specific hybridisation. Stringent in vitro hybridisation conditions are well-known to the person skilled in the art and can be easily taken from the literature (Sambrook et al., (2001), vide supra).

The term “specific hybridisation” refers to the fact that a molecule under stringent conditions binds preferentially to a selected nucleotide sequence, if this nucleotide sequence is part of a complex mixture of, e.g. DNA or RNA molecules. The term “stringent conditions” refers to conditions where a nucleic acid sequence binds preferentially to a target sequence, but not to other sequences, or at least to a significantly lesser extent.

Stringent conditions are also those conditions that allow for the formation of double stranded nucleic acid molecules only if the single stranded sequences provide for high complementarity and preferably for 100% complementarity

According to the invention, two single stranded nucleic acid molecules are highly complementary if the are at least 60%, preferably at least 70%, also preferably at least 80%, particularly preferably at least 90%, also particularly preferably at least 95% and most preferably at least 98% complementary.

Two sequences are considered to be complementary, if the sequences due to their base sequences can build hydrogen bonds between the nucleotides of the single stranded nucleic acid molecules und thus can form duplexes. According to the invention, 100% complementarity between two sequences refers to the situation that every nucleotide of a single stranded nucleic acid molecule base pairs with every base with a nucleotide of another single stranded nucleic acid molecule.

The person skilled in the art knows that the stringency of a hybridisation reaction depends on the temperature during hybridisation and subsequent washing steps as well as on the composition and especially the salt concentrations and the pH-values of hybridisation and washing solutions.

A increased stringency can be generally obtained by using subsequently more diluted salt solutions during hybridisation. An increased stringency may also be achieved by adding destabilising agents such as formamide.

Stringent conditions are e.g. sequence-dependent and will vary dependent on the circumstances. Longer sequences hybridise specifically at higher temperatures. Generally, stringent conditions are chosen, so that temperature for hybridisation is about 5° C. under the thermal melting point (Tm) for the specific sequence at a defined ionic strength and a defined pH. Tm is the temperature (at a defined ionic strength, pH and nucleic acid concentration) where 50% of the molecules complementary to the target sequence hybridise to the target sequence. Typically, stringent conditions are those where the salt concentration comprises between 0.01 to 1.0M sodium ion concentrations (or another salt) and a pH between 7.0 and 8.3. The temperature is at least approx. 30° C. for short molecules (e.g. for those that comprise 10 to 50 nucleotides). Additionally, stringent conditions might be achieved by addition of destabilising agents, such as formamide.

Typical hybridisation buffers are made from 6×SSC, 0,1% SDS (Sambrook et al.(2001), vide supra). Typical Hybridisation temperatures are within 0° C. to 70° C. They do however depend on the length of the hybridising nucleic acid segment and the buffers used. Hybridisation temperature for rather short nucleic acid molecules (18 to 25 nucleotides) are typically between 20° C. to 55° C., preferably between 30° C. and 45° C. and most preferably between 35° C. and 45° C.

Typical washing buffers are made form stock solutions of SSC (Sambrook et al.(2001), vide supra). A typical protocol for the aforementioned rather short molecules comprises three washing steps as e.g. 10 min at 30° C. with 2×SSC, 0,1% SDS, 10 min at 30° C. with 2×SSC and 10 min at 20° C. with 0,2×SSC.

According to the invention, low stringency conditions refer to such reaction conditions whereon the formation of double stranded nucleic acid molecules is allowed for that only have limited complementarity. Tow single stranded nucleic acid molecules show limited complementarity if their sequences are 20%, at most 30%, preferably at most 40%, particularly preferably at most 50% and most preferably at most 60% complementary.

The DNA sequences, which encode a protein having the biological activity of an acyl hydrolase, particularly of a TAG lipase, more preferably of a TAG lipase specific for oxygenated polyene fatty acids, also comprise DNA sequences whose nucleotide sequences are degenerate to one of the DNA sequences described above. The degeneration of the genetic code offers one skilled in the art among other things the possibility of adapting the nucleotide sequence of the DNA sequence to the codon preference (codon usage) of the target plant, i.e. the plant or plant cell which exhibits an altered content of poly-unsaturated fatty acids, particularly of oxygenated polyene fatty acids as a result of the expression of the TAG lipase of the present invention, and thereby optimising the expression.

The above-mentioned DNA sequences also comprise fragments, derivatives and allelic variants of the DNA sequences described above, which code for a protein having the biological activity of an acyl hydrolase, particularly of a TAG lipase, more preferably of a TAG lipase specific for oxygenated polyene fatty acids. The term “fragments” is to be understood as parts of the DNA sequence that are sufficiently long to encode one of the described proteins. The term “derivative” in this context means that the sequences differ from the DNA sequences described above at one or several position/s, but have a high degree of homology to these sequences. Homology here means a sequence identity of at least 25 percent, especially an identity of at least 40 percent, preferably of at least 60 percent, more preferably of more than 80 percent and most preferably of more than 90 percent. The proteins encoded by these DNA sequences show a sequence identity to the amino acid sequences identified in SEQ ID NOs: 2, 4 or 6, respectively, of at least 60 percent, particularly of at least 80 percent, preferably of at least 85 percent and most preferably of more than 90 percent, of more than 95 percent and of more than 98 percent. The differences to the above described DNA sequences may be the result of, for example, deletion, substitution, insertion or recombination.

The DNA sequences that are homologous to the above-mentioned sequences and that are derivatives of these sequences are generally variations of these sequences, which represent modifications that exhibit the same biological function. These variations can be naturally occurring variations, for example sequences from other organisms, or mutations, wherein these mutations can have occurred naturally or have been introduced by targeted mutagenesis. Moreover, the variations may be synthetic sequences. The allelic variants can be naturally occurring variants, synthetic variants or variants created by recombinant DNA techniques.

In a more preferred embodiment the described DNA sequence coding for an acyl hydrolase, particularly for a TAG lipase, more preferably for a TAG lipase specific for oxygenated polyene fatty acids, originates from Arabidopsis thaliana.

The invention also relates to nucleic acid molecules that contain the nucleic acid sequences according to the invention, or which have been occurred therefrom naturally or by gene technological or chemical processes and synthesis methods or which have been derived therefrom. They may be for example DNA or RNA molecules, cDNA, genomic DNA, mRNA and the like.

The invention also relates to such nucleic acid molecules, in which the nucleic acid sequences according to the invention are linked to regulatory elements, which ensure the transcription and, if desired, the translation in the plant cell.

For the expression of the DNA sequences contained in the recombinant nucleic acid molecules according to the invention in plant cells, in principal any promoter can be considered which is active in plant cells. The DNA sequences of the present invention may be expressed in plant cells for example under the control of constitutive regulatory elements, but also under the control of inducible or tissue-specific or development-specific regulatory elements, in particular promoters. While, for example, the use of an inducible promoter allows the purposely triggered expression of the DNA sequences of the present invention in plant cells, the use of, for example, tissue-specific, for example, leaf-specific or seed-specific, promoters offers the possibility to alter the content of oxygenated polyene fatty acids in a certain tissue, for example in leaf or seed tissue. Other suitable promoters mediate e.g. light-inducible gene expression in transgenic plants. With respect to the plants to be transformed the promoter may be homologous or heterologous.

For constitutive expression suitable promoters are e.g. the 35S RNA promoter of the Cauliflower Mosaic Virus and the ubiquitine promoter from maize. The USP promoter (Bäumlein et al. (1991), Mol. Gen. Genet. 225:459-467) or the Hordein promoter (Brandt et al. (1985), Carlsberg Res. Commun. 50:333-345) are examples of useful seed-specific promoters.

Constitutive, germination-specific and seed-specific promoters are preferred within the framework of this invention, as they are especially useful for targeted increasing the content of oxygenated polyene fatty acids in transgenic seeds, also in the context of the anti-sense or co-suppression technique.

In any case the skilled person can find suitable promoters in the literature or can isolate them himself from any desired plant using standard methods.

There are also transcription or termination sequences that provide for correct transcription termination, and may also provide for the addition of a poly(A) tail to the transcript, to the tail being assigned a function in the stabilisation of the transcripts. Such elements are described in the literature (e.g. Gielen (1989) EMBO J. 8:23-29) and are completely interchangeable, e.g. the terminator of the octopin synthase gene from Agrobacterium tumefaciens.

The invention further relates to proteins having the biological activity of an acyl hydrolase, particularly of a TAG lipase, more preferably of a TAG lipase specific for oxygenated polyene fatty acids or biologically active fragments thereof, which are encoded by a nucleic acid sequence of the present invention or a nucleic acid molecule of the present invention. The plant TAG lipase is preferably from Arabidopsis thaliana, more preferably a protein having the amino acid sequence identified in SEQ ID NO:2, SEQ ID NO:4 or SEQ ID NO:6 or an active fragment thereof

Another object of the present invention is to provide vectors whose use facilitates the production of novel plants, in which an altered content of poly-unsaturated oxygenated fatty acids may be achieved. This object is solved by the provision of the vectors of the present invention, which contain nucleic acid sequences that code for enzymes having the activity of an acyl hydrolase, particularly of a TAG lipase, more preferably of a TAG lipase with specificity for oxygenated polyene fatty acids.

The present invention thus also pertains to vectors, especially plasmids, cosmids, viruses, bacteriophages and other vectors, which are commonly used in genetic engineering, which contain the above-mentioned nucleic acid molecules according to the invention and which, optionally, may be used for the transfer of the nucleic acid molecules according to the invention to plants and plant cells.

In a preferred embodiment the nucleic acid molecules contained in the vectors are linked to regulatory elements, which ensure the transcription and, optionally, the translation in prokaryotic and eukaryotic cells.

Optionally, the nucleic acid sequences of the invention may be supplemented with enhancer sequences or other regulatory sequences. These regulatory sequences include for example also signal sequences that ensure the transport of the gene product to a specific compartment.

It is also an object of the present invention to provide novel transgenic plants, plant cells, plant parts, transgenic propagating material and transgenic crop products, which are characterized by a content of polyene fatty acids, particularly of oxygenated polyene fatty acids in the seed oil, that is altered in comparison to wild-type plants or wild-type plant cells.

This problem is solved by the transfer of the nucleic acid molecules of the present invention and their expression in plants. Due to the provision of the nucleic acid molecules according to the invention it is now possible to modify plant cells by gene technological methods in such a manner that they have a novel or altered TAG lipase activity compared to wild-type cells, and as a consequence thereof an alteration in the content of polyene fatty acids, particularly of oxygenated polyene fatty acids in the seed oil is obtained.

Thus, in one embodiment the invention relates to plants and/or their cells, and parts thereof, wherein the content of polyene fatty acids, particularly of oxygenated polyene fatty acids in the seed oil is reduced compared to wild-type plants due to the presence and expression of the nucleic acid molecules according to the invention.

The invention also pertains to those plants, in which the transfer of the nucleic acid molecules of the present invention leads to an increase in the content of polyene fatty acids, particularly of oxygenated polyene fatty acids in the seed oil. Such a reduction may be achieved for example by the transfer of anti-sense constructs or by other suppression mechanisms, such as for example co-suppressions.

The invention further relates to transgenic plant cells and plants comprising said plant cells, and parts and products of these plants, which contain the nucleic acid molecules according to the invention integrated into the plant genome. Further, the invention relates to plants, whose cells contain the nucleic acid sequence of the present invention in self-replicating form, i.e. the plant cell contains the foreign DNA on a separate nucleic acid molecule (transient expression).

The plants, which are transformed with the nucleic acid molecules according to the invention and in which an altered amount of polyene fatty acids, particularly of oxygenated polyene fatty acids in the seed oil is produced due to the introduction of such a molecule, may in principle be any plant. Preferably it is a monocotyledonous or dicotyledonous useful plant.

Examples for monocotyledonous plants are plants belonging to the genus of Avena (oats), Triticum (wheat), Secale (rye), Hordeum (barley), Oryza (rice), Panicum, Pennisetum, Setaria, Sorghum (millet), Zea (maize). Examples of dicotyledonous useful plants include, inter alia, Leguminosae, such as legumes (leguminous plants) and particularly alfalfa, soybean, rape, tomato, sugar beet, potato, ornamental plants or trees. Other useful plants may for example include fruits (in particular apples, pears, cherries, grapes, citrus, pineapple and banana), palm trees; tea, cocoa and coffee trees; tobacco, sisal, cotton, flax, sunflower as well as officinal (medical) herbs and grass from pasture as well as forage plants. More preferred are the cereals wheat, rye, oats, barley, rice, maize and millet, forage cereal, sugar beet, rapeseed, soybean, tomato, potato, Poales (sweet grass), feed grasses and clover. It goes without saying that the invention especially pertains to common food or forage plants. These include, beside the other plants already mentioned above, peanut, lentil, Vicia faba, Beta vulgaris, buckwheat (Fagopyrum sagittatum), carrot, sunflower, topinambur, Brassica rapa, white cabbage, rape seed and turnip seed.

More preferred are oil seed plants, i.e. plants whose seeds contain oil.

The present invention also relates to the seed oil which can be isolated from the transgenic plant cells or plants according to the invention by the usual methods known to the person skilled in the art. Such seed oils are particularly suitable for the food industry because of the decreased amount of polyene and oxygenated polyene fatty acids which provides for a better storage stability of these seed oils compared to conventional seed oils. The invention therefore also relates to the use of the transgenic plant cells and plants according to the invention for producing seed oils with decreased amounts of polyene and oxygenated polyene fatty acids.

The present invention also relates to propagating material and crop products of the plants according to the invention such as seeds, fruits, cuttings, rhizomes/rootstocks etc., as well as parts of said plants such as protoplasts, plant cells and calli.

In a further embodiment the invention pertains to host cells, especially prokaryotic and eukaryotic cells, which have been transformed, or infected, with a nucleic acid molecule mentioned above or a vector, as well as cells that are derived from such host cells and contain the described nucleic acid molecules or vectors. The host cells may be bacteria, viruses, algae, yeast and fungal cells as well as plant or animal cells.

The invention also relates to such host cells that besides the nucleic acid molecules of the present invention further contain one or more nucleic acid molecules transferred by means of gene technology or in a natural way, which carry the genetic information for enzymes involved in the LOX dependent catabolism of poly-unsaturated fatty acids in plants.

A further object of the present invention is to provide methods of producing plant cells and plants, which exhibit an altered content of polyene fatty acids, especially of oxygenated polyene fatty acids, in the seed oil.

This problem is solved by methods, which facilitate the production of novel plant cells and plants having an altered content of polyene fatty acids, especially of oxygenated polyene fatty acids, in the seed oil due to the transfer of the nucleic acid molecules according to the invention which code for acyl hydrolase, especially for TAG lipase. Various methods may be used for producing such novel plant cells and plants. On one hand, plants or plant cells may be modified using conventional gene technological transformation methods in such a way that the novel nucleic acid molecules are integrated into the plant genome, i.e. resulting in stable transformants. On the other hand, a nucleic acid molecule of the present invention, whose presence and, optionally, expression in the plant cell results in an altered biosynthetic performance, may be contained as a self-replicating system in the plant cell or plant. The nucleic acid molecules of the invention may, for example, be contained in a virus that the plant or plant cell comes in contact with.

According to the invention, plants and plant cells that have an altered content of polyene fatty acids, especially of oxygenated polyene fatty acids, in the seed oil due to the expression of a nucleic acid sequence according to the invention are produced by a method comprising the following steps:

a) producing a recombinant nucleic acid molecule comprising the following components in 5′-3′-orientation:

regulatory sequences of a promoter that is active in plants,

operatively linked thereto a nucleic acid sequence, which codes for a protein having the biological activity of an acyl hydrolase, especially of a TAG lipase, more preferably of a TAG lipase with specificity for oxygenated polyene fatty acids, and

optionally, operatively linked thereto sequences, which may act as transcription, termination and/or polyadenylation signals in plant cells.

b) transferring the nucleic acid molecule from a) to plant cells.

Alternatively, one or more nucleic acid sequences of the present invention may be introduced into the plant cell or plant as a self-replicating system.

As a further alternative, step a) of the above method may be modified in that the nucleic acid sequence according to the invention, which encodes a protein having the biological activity of an acyl hydrolase, especially of a TAG lipase, more preferably of a TAG lipase which is specific for oxygenated polyene fatty acids, is linked to the 3′-end of the promoter in anti-sense orientation.

In another aspect of the present invention one or more additional nucleic acid molecules, which code for proteins that catalyse the transfer of fatty acids to glycerides (transacylases), may also be introduced. Thus, lipids whose oxygenated polyene fatty acids have been cleaved by the TAG lipase of the invention may be substituted with non-oxygenated poly-unsaturated fatty acids, which are for example suitable for the use of the resulting plant oil in the food industry.

In order to prepare the introduction of foreign genes into higher plants, or their cells, a large number of cloning vectors are available which contain a replicating signal for E. coli and a marker gene for selecting transformed bacterial cells. Examples for such vectors are pBR322, pUC series, M13mp series, pACYC184 etc. The desired sequence may be introduced in the vector at a suitable restriction site. The resulting plasmid is then used for the transformation of E. coli cells. Transformed E. coli cells are cultivated in a suitable medium and then harvested and lysed, and the plasmid is recovered. In order to characterise the produced plasmid DNA in general restriction analyses, gel electrophoreses and other biochemical and molecular biological methods are used as the analytic method. After each manipulation step the plasmid DNA may be cleaved and the obtained DNA fragments may be linked to other DNA sequences.

A precondition for the introduction of the recombinant nucleic acid molecules and vectors of the invention into plant cells is the availability of suitable transformation systems. During the last two decades a wide range of transformation methods have been developed and established. These techniques comprise the transformation of plant cells with T-DNA by use of Agrobacterium tumefaciens or Agrobacterium rhizogenes as the transforming agent, the diffusion of protoplasts, the direct gene transfer of isolated DNA into protoplasts, the microinjection and electroporation of DNA into plant cells, DNA introduction by means of the biolistic methods as well as further possibilities. The person skilled in the art will have no difficulty in selecting the suitable method. All transformation techniques have been well established for many years and belong unquestionably to the standard repertoire of one skilled in the art who is familiar with the molecular biology of plants, plant biotechnology, and cell and tissue cultivation.

During the injection and electroporation of DNA into plant cells no specific requirements for the used plasmids are necessary per se. The same is true for the direct gene transfer. Simple plasmids such as pUC-derivatives may be used. But the presence of a selectable marker gene is advisable if entire plants are to be regenerated from such transformed cells. The person skilled in the art is familiar with these common selection markers and he will not have any problems in selecting a suitable marker.

Further DNA sequences may be required depending on the introduction method for desired genes into the plant cell. If, for example, the Ti or Ri plasmid is used for the transformation of the plant cell, at least the right border, however more often both the right and the left border of the T-DNA in the Ti or Ri plasmid has to be linked as flanking region to the genes to be introduced. If agrobacteria are used for the transformation, the DNA to be introduced has to be cloned into special plasmids, either into an intermediate or into a binary vector. Intermediate vectors may be integrated into the Ti or Ri plasmid of the agrobacteria by homologous recombination because of sequences, which are homologous to sequences in the T-DNA. This also contains the vir region that is required for T-DNA transfer. Intermediate vectors are not able to replicate in agrobacteria. With the aid of a helper plasmid, the intermediate vector may be transferred to Agrobacterium tumefaciens (conjugation). Binary vectors are able to replicate in E. coli as well as in agrobacteria. They contain a selection marker gene, and a linker or polylinker framed by the right and left T-DNA border region. They may be transformed directly into agrobacteria. The agrobacterial host cell should contain a plasmid with a vir region. The vir region is required for the transfer of the T-DNA into the plant cell. Additional T-DNA may be present. Such a transformed agrobacterial cell is used for the transformation of plant cells. The use of T-DNA for the transformation of plant cells has been studied intensively, and has been sufficiently described in generally known reviews and plant transformation manuals.

For the transfer of the DNA into the plant cell, plant explantates may be cultivated for this purpose together with Agrobacterium tumefaciens or Agrobacterium rhizogenes. From the infected plant material (e.g. leaf pieces, stem segments, roots, but also protoplasts or suspension-cultivated plant cells) whole plants may again be regenerated in a suitable medium, which may contain antibiotics or biocides for the selection of transformed cells.

Once the introduced DNA has been integrated into the plant cell genome it is generally stable there and also remains in the progeny of the originally transformed cell. It normally contains a selection marker that makes the transformed plant cells resistant to a biocide or an antibiotic, such as kanamycin, G418, bleomycin, hygromycin, methotrexate, glyphosate, streptomycin, sulfonylurea, gentamycin or phosphinotricin and others. The individually selected marker should therefore allow the selection of transformed cells from cells lacking the introduced DNA. Alternative markers are suitable here, such as for example nutritive markers, screening markers (such as GFP, green fluorescent protein). Of course, it could also be carried out without any selection markers, although this would involve quite intensive screening efforts. If the selection marker used is to be removed after having successfully transformed the cells or plants and after having identified the successful transformation, the person skilled in the art has various strategies at his disposal. For example sequence specific recombinases may be used e.g. in form of retransformation of a recombinase expressing stock line and outcrossing the recombinase after succeeding in the removal of the selection marker (see e.g. Reiss et al. (1996) Proc. Natl. Acad. Sci. USA 93:3094-3098; Bayley et al. (1992) Plant. Mol. Biol 18:353-361; Lloyd et al. (1994) Mol. Gen. Genet. 242:653-657; Mäser et al. (1991) Mol. Gen. Genet. 230:170-176; Onouchi et al. (1991) Nucl. Acids Res. 19:6373-6378). The selection marker also may be removed by co-transformation followed by outcrossing.

Regeneration of the transgenic plants from transgenic plant cells is carried out according to conventional regeneration methods using general media and auxines. The plants thus obtained may then, optionally, be identified by conventional methods including molecular biological methods such as PCR, blot analyses, or biochemical techniques for the presence of the introduced DNA, which encodes a protein having the enzymatic activity of a TAG lipase that is specific for oxygenated polyene fatty acids, or for the presence of TAG lipase enzyme activity.

Independent of the used regulatory sequences which control the expression of the acyl hydrolase sequences of the invention, especially TAG lipase DNA sequences, the person skilled in the art has a wide range of molecular biological and/or biochemical methods at his disposal for analysing the transformed plant cells, transgenic plants, plant parts, crop products and propagating material for usage such as for example PCR, Northern Blot analysis for detecting RNA specific for the TAG lipase of the invention or for determining the accumulated amount of the TAG lipase specific RNA, Southern Blot analysis for identifying DNA sequences according to the invention encoding the TAG lipase or Western Blot analysis for detecting the TAG lipase encoded by the nucleic acid molecules of the present invention. Of course, the person skilled in the art, using protocols obtained in literature, may determine the enzymatic activity of the TAG lipase according to the invention. Moreover, one can, for example, place seeds, which are obtained by self-crossing or crossbreeding, on a medium containing a selective agent that matches the selection marker transferred in connection with the TAG lipase DNA sequence, and based on the germinability and the growth of the filial generation(s) and the segregation pattern, may draw conclusions about the genotype of the corresponding plant.

It is another object of the invention to point out possible applications of the nucleic acid sequences of the invention as well as the nucleic acid molecules in which they are contained.

This problem is solved by the inventive applications of the novel DNA sequences and molecules for producing plant cells and plants, characterised by an altered content especially of polyene fatty acids, more preferably of oxygenated polyene fatty acids, in the seed oil compared to wild-type cells or wild-type plants.

The present invention further comprises any feasible form of using the nucleic acid molecules according to the invention, whose presence and, optionally, expression in plants effects an alteration in the content of polyene fatty acids, especially of oxygenated polyene fatty acids in the seed oil as well as any feasible form of using the proteins according to the invention or fragments thereof, whose enzymatic activity brings about such an alteration.

The invention further concerns the use of a DNA sequence of the present invention or parts thereof for identifying and isolating homologous sequences from plants or other organisms.

The inventive nucleic acid molecules may thus be used according to the invention for producing transgenic plants containing a higher or lower content of the inventive acyl hydrolases, especially TAG lipases, than occurs naturally, or is present in cell types or developmental stages, where the inventive acyl hydrolases, especially TAG lipases, are normally not found. This causes an alteration in the content of triacylglycerol and cholesteryl esters in these cells.

Accumulation of fatty acids with unusual structures may be an advantageous phenotype in plants used for foodstuffs. Triacylglycerol lipases may be also useful in processing plant oils and in the development of novel seed oils, since storage stability or shelf life of the plant seed oils for the food industry may be prolonged due to a selective cleavage of oxygenated polyene fatty acid residues by the activity of the inventive TAG lipases.

For many applications it can be useful to introduce the inventive TAG lipases into different cellular compartments or to facilitate their secretion from the cell. Thus, it is possible to modify the nucleic acid molecules according to the invention to such an extent that the nucleic acid molecules of the invention are supplemented with suitable intracellular target sequences such as transit sequences (K. Keegstra (1989) Cell 56:247-253), signal sequences and the like.

For many applications it may also be desired to reduce or eliminate the expression of nucleic acid molecules coding for TAG lipases, especially for TAG lipases that are specific for oxygenated polyene fatty acids. For this purpose a nucleic acid molecule developed for the co-suppression of the inventive TAG lipase may be created by linking a gene or gene fragment encoding a TAG lipase specific for oxygenated polyene fatty acids to plant promoter sequences. Alternatively, a nucleic acid molecule developed for the expression of anti-sense RNA for the entire or part of the inventive nucleic acid molecule may be created by linking the gene or gene fragment in reverse orientation to plant promoter sequences. Both the genes for the co-suppression and the anti-sense genes can be introduced into the plants by transformation, resulting in a reduction or elimination of the expression of the corresponding endogenous gene.

The invention is based on the successful isolation of cDNA clones encoding an acyl hydrolase, especially a TAG lipase, more preferably a TAG lipase that is specific for oxygenated polyene fatty acids, from a cDNA library of Arabidopsis thaliana. The sequences of these cDNA clones comprising a complete reading frame are identified in SEQ ID NO: 1, SEQ ID NO: 3 and SEQ ID NO: 5. Using these sequences, it was possible for the first time to produce transgenic plants which, due to the transfer and expression of a TAG lipase, especially a TAG lipase specific for oxygenated polyene fatty acids, exhibit an altered content in oxygenated polyene fatty acids in the seed oil compared to wild-type plants.

The following examples are intended to illustrate the present invention and are in no way to be understood as limiting.

### EXAMPLES

### Example 1

Cloning and Expression of a cDNA Encoding the TAG Lipase from Arabidopsis Thaliana

cDNA sequences encoding a TAG lipase from Arabidopsis thaliana were identified by screening Arabidopsis thaliana cDNA libraries produced as described below according to a standard protocol, also described below. Three of the annotated sequences (lipase 1: SEQ ID NO: 1, lipase 2: SEQ ID NO: 3, lipase 3: SEQ ID NO: 5) were cloned, expressed and tested for their activity.

Production of Different CDNA Libraries from Arabidopsis Thaliana

According to the protocol described below cDNA libraries were produced from a mixture of various organs of Arabidopsis thaliana as well a cDNA library from blossoms of Arabidopsis thaliana.

RNA Isolation from Arabidopsis

RNA isolation from Arabidopsis was performed using the following reagents:

extraction buffer I:

100 mM Tris/HCl pH 7.5

25 mM EDTA

2% lauryl sarcosyl

4 M guanidinium thiocyanate

5% (w/v) PVP (Polyklar) (is added as insoluble substance to the prepared solution only at the beginning of the experiment)

1 ml/100 ml β-mercaptoethanol (is added just before use of the extraction buffer)

PCI:

20 ml phenol

20 ml chloroform

1 ml isoamyl alcohol (saturated with H₂O)

chloroform

70% ethanol, 100% ethanol

DEPC treated H₂O

extraction buffer II (prepared using DEPC treated water):

100 ml Tris/HCl pH 8.8

100 mM NaCl

5 mM EDTA

2% SDS

The isolation of RNA from Arabidopsis is carried out according to the following protocol:

triturate 20 g plant material in liquid nitrogen

prepare 100 ml extraction buffer I with PVP and mercaptoethanol, add the triturated plant material and mix immediately

after homogenisation the solution is transferred to Falcon tubes and is shaken for approximately 15 minutes

centrifuge for 10 to 15 minutes at 4,500 to 5,500 rpm in Falcon tubes in a sigma centrifuge

transfer the supernatant into fresh Falcons and discard the pellets

mix with 1 volume PCI and shake for 15 minutes, allow to stand on ice for 15 minutes and then centrifuge, remove the supernatant and transfer it into fresh Falcons

remove the supernatant and mix it with 1 volume chloroform, shake for 15 minutes and centrifuge

remove the supernatant and add 1 volume isopropanol, precipitate overnight at −20° C.

centrifuge for 30 minutes at 9,000 rpm

dissolve each of the pellets in 20 ml extraction buffer II and add 1 volume isopropanol

incubate for 1 hour at −20° C.

centrifuge for 30 minutes at 9,000 rpm

rinse the pellets twice with 1 ml 70% ethanol

dry the pellets in a speed-vac

dissolve the pellets in 1500 ml H₂O, allow to stand on ice for 30 minutes for dissolving it, remove the supernatant and transfer it into fresh Eppendorf caps, allow to stand cold

Measure the RNA in a photometer in a dilution of 1:100, during measurement the ratio of A₂₆₀ to A₂₈₀ should be greater than/equal to 1.8. The measured value is multiplied with the dilution factor and a factor of 40, thus leading to the RNA concentration in μl/ml. Performance of a wavelength scan of 200-300 nm results in a typical RNA curve with a maximum at 260 nm. The total amount of RNA is then calculated and an RNA agarose gel is run in order to determine the quality of the RNA.

b) mRNA Isolation

Isolation of mRNA is performed with the mRNA isolation kit poly-Attract obtainable from Promega (Mannheim, Germany).

c) RT-PCR

The reverse transcriptase PCR was done with 1 μg RNA, 200 pmol oligo-dT with Superscript II from Gibco (Eggenstein, Germany) according to the manufacture's protocol.

II. PCR Amplification

The PCR amplification was prepared with the Expand™ High Fidelity PCR system according to the manufacture's protocol (Boehringer Mannheim, Germany). The cDNA mixture of Arabidopsis was used as template for the lipases 1 and 2 and the cDNA from the blossom of Arabidopsis was used for the lipase 3, respectively.

lipase 1: (3× methionine within the starter
region of the sequence)
5′:-CCC GCA TGC ATG CAG TTG TCT CCG GAA CGA TGC

(A1)

5′:-GGG GCA TGC ATG TCT GAA AAC AGA GAG GCT TGG

(A2)

5′:-AAA GCA TGC ATG GAG CTG CTT CAC GGC TCC AAC

(A3)

3′:-AAA GTC GAC TCA AAA AGG GCT GAC CCT GCC AGC C
lipase 2:
5′:-CCC CGC ATG CAT GGC TTC TTC ACT GAA GAA
3′:-AAA CTG CAG TTA TGT ATC CAC TGT ACC AGA GCC

AAG

lipase 3:
5′:-GGG GCA TGC ATG TGT AGA AGA TAT TTC GTG CAT

AGT

3′:-TTT CTG CAG TGA CAG ATG AGG TTT GCC TGT TTC C

For the production of the expression clones, which over-express the recombinant TAG lipase in E. coli, the open reading frame for the entire unprocessed protein was PCR amplified using the above-mentioned oligonucleotide primers of the DNA sequences according to the sequences identified in SEQ ID NOs: 1, 3 and 5. The conditions for the PCR reaction were as follows: denaturation for 2 minutes at 94° C., then for 10 cycles: denaturation for 30 seconds at 94° C., annealing for 30 seconds at 65° C., elongation for 3 minutes at 72° C., then for 15 cycles: denaturation for 30 seconds at 94° C., annealing for 30 seconds at 65° C., elongation for 3 minutes at 72° C. plus a time increment of 5 seconds per each cycle, to the end an elongation step for another 2 minutes at 72° C.

The amplified PCR fragment was ligated into the expression vector QIAexpress pQE 30 and transformed as a pre-clone into XL1-Blue cells using the pGEM^{R}-T Easy vector system II kit (Promega, Madison, USA). In the following sub-cloning was carried out in the expression strain E. coli SG13009[pREP4].

IV. For the expression of plant TAG lipase the E. coli strain was cultivated in LB medium at 10° C. for a period of 72 hours.

V. Purification of Membrane-Associated Proteins

Purification was carried out according to the following steps:

producing a membrane fraction of the E. coli SG13009[pREP4]

solubilizing in sodium phosphate pH 8.0, 1 M NaCl, 0.5% Triton

centrifuging for 1 hour at 4° C. and 37,000 rpm

purifying the supernatant using a Talon metal affinity resin (Clontech, Palo Alto, USA) according to the manufacture's instructions

eluting the protein in sodium phosphate, 1M NaCl at pH 5.0

### Example 2

Evaluating the TAG Lipase Activity

In order to evaluate the TAG lipase activity the eluted protein fraction obtained, as described above, was incubated with a lipid extract (chloroform:methanol, Bligh/Dyer 1959), which was isolated from 4 day old seedlings of Cucumis sativa, for 40 minutes at 25° C. and a buffer concentrate (1M Tris pH 8.5; 0.5 M NaCl; 50 mM CaCl₂) was added to adjust the pH to 8.0. The mixture was acidified with acetic acid and was extracted with hexane. Analysis was carried out with HPLC (LiCHrosphere 100 RP-18 (Merck, Darmstadt, Germany); the elution agent was: acetonitrile:H₂O acetic acid 70:30:0.1).

### Example 3

Transformation of Arabidopsis thaliana and Nicotiana tabacum Plants and Regeneration of Intact Plants

In order to produce transgenic plants over-expressing the TAG lipase sequence, which therefore have an increased activity of the enzyme TAG lipase compared to non-transformed plants, each of the DNA sequences identified in SEQ ID NOs: 1, 3 and 5 was cloned into a suitable binary vector (e.g. pPCV001, pPCV002 (Koncz & Schell 1986 MGG 204:383-396).

Instead of the vector mentioned any vector suitable for plant transformation may be used for the production of a chimeric gene consisting of a fusion of the CaMV 35S promoter or another constitutive, seed-specific, germination-specific or blossom-specific promoter that ensures transcription and, if desired, translation in plant cells, and DNA sequences encoding TAG lipase.

The binary vector was then transformed into Agrobacterium tumefaciens (strain GV2260; Horsch et al. (1985), Science 227:1229-1231) and used to transform Arabidopsis and tobacco plants (SNN) using the leaf disc transformation technique (Horsch et al., supra).

For this an overnight culture of the respective Agrobacterium tumefaciens clone was centrifuged for 10 minutes at 5,000 rpm, and the bacteria were resuspended in 2YT medium. Leaf pieces of sterile cultures of Arabidopsis thaliana or Nicotiana tabacum cv. Samsun NN were placed in a bacterial suspension for a short while. The leaf pieces were then laid on MS medium (Murashige and Skoog (1962), Physiol. Plant. 15:473; 0.7% agar) and incubated for two days in dark. For shoot induction the leaf pieces were then laid on MS medium (0.7% agar) containing 1.6% glucose, 1 mg/l 6-benzylamino purine, 0.2 mg/l naphthyl acetic acid, 500 mg/l claforan (cefotaxim, Hoechst, Frankfurt) and 50 mg/l kanamycin. The medium was changed every seven to ten days. After shoots had developed the leaf pieces were transferred into glass jars containing the same medium. Shoots were cut off as they appeared and were laid on MS medium containing 2% sucrose and 250 mg/l claforan and grown until entire plants were regenerated.

Analysis of the resulting transgenic Arabidopsis and tobacco plants confirmed the successful alteration in the lipid content due to expression of the nucleic acid molecules of the present invention.

1. A method of producing plants or plant cells, respectively, which have an altered content of unsaturated fatty acids and particularly of oxygenated polyene fatty acids, comprising the following steps,
a) producing a nucleic acid sequence comprising the following components, which are successively arranged in 5′-3′-orientation:
a promoter that is functional in plants and particularly seed-specific,
at least one DNA sequence that codes for a protein having the enzymatic activity of a TAG lipase, more preferably of a TAG lipase which is specific for oxygenated polyene fatty acids, selected from the group consisting of:
i. DNA sequences comprising the nucleotide sequence identified in SEQ ID NO: 1,
ii. DNA sequences comprising the nucleotide sequence identified in SEQ ID NO: 3,
iii. DNA sequences comprising the nucleotide sequence identified in SEQ ID NO: 5,
iv. DNA sequences that encode the amino acid sequence identified in SEQ ID NO: 2 or fragments thereof,
v. DNA sequences that encode the amino acid sequence identified in SEQ ID NO: 4 or fragments thereof,
vi. DNA sequences that encode the amino acid sequence identified in SEQ ID NO: 6 or fragments thereof,
vii. DNA sequences comprising a nucleotide sequence having a sequence identity of at least 60%, preferably of more than 80% and more preferably of more than 90% to the sequences mentioned in i. to vi.,
viii. DNA sequences comprising one of the sequences mentioned in i. to vii. in anti-sense orientation,
b) transferring the nucleic acid from a) to plant cells and, optionally, integrating the nucleic acid sequence into the plant genome.

2. The method according to claim 1, characterised in that the promoter is a constitutive promoter, preferably the 35S RNA promoter of the CaMV.

3. The method according to claim 1 or 2, characterised in that the DNA sequences mentioned in claim 1 are part of a vector.

4. A transgenic plant cell, produced by a method according to any of the claims 1 to 3.

5. The plant cell according to claim 4, containing at least one further foreign gene.

6. The plant cell according to claim 4 or 5, including protoplasts, which compared to non-transformed plant cells has an altered, especially increased content of TAG lipase, especially an increased content of TAG lipase that is specific for oxygenated polyene fatty acids.

7. A transgenic plant containing a plant cell according to any of the claims 4 to 6 or produced according to any of the claims 1 to 3, as well as parts of said plants, transgenic crop products and transgenic propagating material of said plants, such as protoplasts, plant cells, calli, seeds, rhizomes/rootstocks, cuttings, as well as the transgenic progenies of said plants.

8. The plants according to claim 7, which compared to wild-type plants have an altered content of TAG lipase, especially an altered content of TAG lipase that is specific for oxygenated polyene fatty acids.

9. The plants according to claim 8, which compared to wild-type plants have an increased content of TAG lipase, especially an increased content of TAG lipase that is specific for oxygenated polyene fatty acids.

10. The plants according to any of the claims 7 to 9, which compared to wild-type plants contain an altered content of polyene fatty acids, especially of oxygenated polyene fatty acids, in the seed oil.

11. The plants according to claim 10, which compared to wild-type plants have a reduced content of polyene fatty acids, especially of oxygenated polyene fatty acids, in the seed oil.

12. Dicotyledonous plants according to any of the claims 7 to 11.

13. The plants according to claim 12, which are useful plants, food plants and/or forage plants.

14. Monocotyledonous plants according to any of the claims 7 to 11.

15. The plants according to claim 14, which are useful plants, food plants and/or forage plants.

16. Use of a plant according to any of the claims 7 to 15 as useful plants, food plants and/or forage plants.

17. Use of a nucleic acid sequence, selected from the group consisting of:
i. DNA sequences comprising the nucleotide sequence identified in SEQ ID NO: 1,
ii. DNA sequences comprising the nucleotide sequence identified in SEQ ID NO: 3,
iii. DNA sequences comprising the nucleotide sequence identified in SEQ ID NO: 5,
iv. DNA sequences encoding the amino acid sequence identified in SEQ ID NO: 2 or fragments thereof,
v. DNA sequences encoding the amino acid sequence identified in SEQ ID NO: 4 or fragments thereof,
vi. DNA sequences encoding the amino acid sequence identified in SEQ ID NO: 6 or fragments thereof,
vii. DNA sequences comprising a nucleotide sequence that has a sequence identity of at least 60%, preferably of more than 80% and more preferably of more than 90% to the sequences mentioned in i. to vi.,
viii. DNA sequences comprising one of the sequences mentioned in i. to vii. in anti-sense orientation,
for producing transgenic plants or plant cells, respectively, having an altered content of unsaturated fatty acids, particularly of oxygenated polyene fatty acids, particularly in the seed oil.

18. Use according to claim 17, wherein the plants or plant cells, respectively, have an altered content of TAG lipase, especially an altered content of a TAG lipase that is specific for oxygenated polyene fatty acids.

19. A method of altering the content of unsaturated fatty acid residues, especially of oxygenated polyene fatty acid residues, in transgenic plants by influencing the lipoxygenase(LOX)-dependent metabolism of poly-unsaturated fatty acids in transgenic plants.

20. The method according to claim 19, wherein the LOX-dependent metabolism of poly-unsaturated fatty acids is altered by modifying the expression of TAG lipases, comprising the steps:
a) optionally, transforming a host cell with LOX,
b) transforming a host cell with a nucleic acid molecule, selected from the group consisting of:
i. DNA sequences comprising the nucleotide sequence identified in SEQ ID NO: 1,
ii. DNA sequences comprising the nucleotide sequence identified in SEQ ID NO: 3,
iii. DNA sequences comprising the nucleotide sequence identified in SEQ ID NO: 5,
iv. DNA sequences that encode the amino acid sequence identified in SEQ ID NO: 2 or fragments thereof,
v. DNA sequences that encode the amino acid sequence identified in SEQ ID NO: 4 or fragments thereof,
vi. DNA sequences that encode the amino acid sequence identified in SEQ ID NO: 6 or fragments thereof,
vii. DNA sequences comprising a nucleotide sequence that has a sequence identity of at least 60%, especially of more than 80% and more particularly of more than 90% to the sequences mentioned in i. to vi.,
viii. DNA sequences comprising one of the DNA sequences mentioned in i. to vii. in anti-sense orientation,
c) cultivating the transformed host cells produced in step a) and b) under conditions that are suitable for expressing the nucleic acid molecule mentioned in b).

21. Use of a TAG lipase, especially of a TAG lipase that is specific for oxygenated polyene fatty acids and that is encoded by a nucleic acid molecule, selected from the group consisting of:
i. DNA sequences comprising the nucleotide sequence identified in SEQ ID NO: 1
ii. DNA sequences comprising the nucleotide sequence identified in SEQ ID NO: 3,
iii. DNA sequences comprising the nucleotide sequence identified in SEQ ID NO: 5,
iv. DNA sequences that encode the amino acid sequence identified in SEQ ID NO: 2 or fragments thereof,
v. DNA sequences that encode the amino acid sequence identified in SEQ ID NO: 4 or fragments thereof,
vi. DNA sequences that encode the amino acid sequence identified in SEQ ID NO: 6 or fragments thereof,
vii. DNA sequences comprising a nucleotide sequence that has a sequence identity of at least 60%, preferably of more than 80% and more preferably of more than 90% to the sequences mentioned in i. to vi.,
for processing plant oils, which comprise oxygenated polyene fatty acids.

22. Use according to claim 21 for specifically isolating the oxygenated polyene fatty acid residues from plant oils.

23. Use according to claim 21 or 22 for reducing the content of oxygenated polyene fatty acid residues in plant oil.

24. A seed oil derived from transgenic plants and/or plant cells according to claims 4 to 15.

25. Use of transgenic plants and/or plant cells according to claims 4 to 15 for producing and/or isolating the seed oil of such plants.
